(19) United States
(12) Patent Application Publication
(10) Pub. No.: US 2021/0038245 A1
(43) Pub. Date: Feb. 11, 2021

(54) EPIDERMAL HARVESTER WITH INTEGRATED MECHANICAL STRETCHING MECHANISM

(71) Applicant: KCI Licensing, Inc., San Antonio, TX (US)

(72) Inventors: Sandra Osborne GARCIA, San Antonio, TX (US); Tyler H. SIMMONS, San Antonio, TX (US); David Richard MERCER, Bournemouth (GB)

(21) Appl. No.: 16/965,973

(22) PCT Filed: Jan. 29, 2019
(86) PCT No.: PCT/US2019/015504

§ 371 Date: Jul. 29, 2020

Related U.S. Application Data
(60) Provisional application No. 62/623,404, filed on Jan. 29, 2018.

(51) Int. Cl. A61B 17/322; A61F 13/00; A61M 1/00

(52) U.S. Cl. CPC A61B 17/322; A61B 2017/0042; A61M 1/0086; A61F 13/00

(57) ABSTRACT

A device for generating skin grafts is disclosed, which includes a harvester having a distal end for placement on or in proximity of a portion of a subject's skin, where the harvester includes an orifice plate coupled to the distal end and having one or more openings. The device further includes a lid that is configured for attachment to the harvester to form a chamber and is configured for coupling to a vacuum source for generating a negative pressure in said chamber, thereby raising at least one blister through said at least one opening of the orifice plate. A skin-stretching mechanism is coupled to the harvester for stretching said skin portion so as to facilitate formation of said at least one blister. The harvester further includes a cutter integrated therein for cutting the blister. In some embodiments, the skin-stretching mechanism is coupled to the distal end of the harvester.

### TECHNICAL FIELD

The present invention relates generally to devices and methods for generating skin grafts, and more particularly to such devices and methods that provide stretching of a skin portion to allow more facile generation of micrograft blisters.

### BACKGROUND

Skin grafting is a surgical procedure in which a section of skin is removed from one area of a person's body (autograft), removed from another human source (allograft), or removed from another animal (xenograft), and transplanted to a recipient site of a patient, such as a wound site. Chronic wounds are often observed in elderly patients and/or in patients with severe comorbidities. A common feature of different types of chronic wounds is impaired re-epithelization. Autologous epidermal grafting provides a solution to wound closure by transferring the patient's own epidermal cells to the wound site.

In one technique, a plurality of epidermal microblisters are generated by raising a plurality of blisters through openings of an orifice plate attached to an end of a low-pressure chamber. The raised blisters can be harvested and transplanted onto a wound site. A device marketed by the assignee of the present application under the trade designation CelluTome® can be used to create and harvest such epidermal microblisters in an automated and precise manner.

There is still a need for improved devices and methods that can allow for faster generation of skin grafts and hence a lower risk of complications and a more efficient workflow for the medical practitioners.

### SUMMARY

The present disclosure relates to methods, apparatus, and systems for harvesting skin grafts. In one aspect, a skin-graft harvester can be disposed adjacent to a portion of a subject's skin, where the harvester includes an orifice plate coupled to the distal end and having one or more openings. The device further includes a lid that is configured for attachment to the harvester to form a chamber and is configured for coupling to a vacuum source for generating a negative pressure in said chamber, thereby raising at least one blister through said at least one opening of the orifice plate. A skin-stretching mechanism is coupled to the harvester for stretching said skin portion, e.g., so as to facilitate formation of said at least one blister. The harvester further includes a cutter integrated therein for cutting the blister. In some embodiments, the skin-stretching mechanism is coupled to the distal end of the harvester.

In a related aspect, a method for generating a skin graft is disclosed, which includes stretching a portion of a subject's skin, and placing a distal end of a device configured to generate one or more blisters adjacent to the skin portion, wherein the device comprises a negative pressure chamber and an orifice plate fitted to a distal end of the chamber and having one or more openings through which skin blisters can be raised into an interior of the chamber, and a cutter integrated within the chamber for cutting the blisters, and applying a negative pressure to said chamber to raise one or more blisters through one or more openings of the orifice plate while maintaining the skin portion in a stretched state. The cutter of the blister-generating device can be activated to cut the blisters. The cut blisters can be transferred to a dressing, e.g., a silicone dressing. In some embodiments, the blisters can have a lateral dimension less than about 2 mm, e.g., in a range of about 100 to about 200 microns.

In other examples, any of the aspects above, or any system, method, apparatus described herein can include one or more of the following features.

A variety of skin-stretching mechanisms can be employed. For example, in some embodiments, the skin-stretching mechanism includes a pair of slide rollers disposed on opposed sides of the orifice plate such that rotation of the slide rollers in opposite directions stretches the skin portion disposed below the orifice plate. In some such embodiments, the slide rollers can include a tacky skin-contacting surface that is configured for placement on a subject's skin. By way of example, the tacky surface can be formed of a biocompatible material. Some examples of suitable materials can include, without limitation silicon, acrylic, or a mix of silicone and acrylic in a pattern. Generally, any suitable tacky and/or biocompatible material available in the art can be used.

In some embodiments, each of the slide rollers can include a finger-grip protrusion to allow manual rotation thereof. By way of example, each of the slide rollers can have a cylindrical shape extending between a proximal end and a distal end along a portion of a bottom surface of the harvester. A finger-grip protrusion can be provided at one of the proximal or distal ends of each roller to allow manual rotation thereof.

In some embodiments, the skin-stretching mechanism can include a pair of adhesive strips disposed on a bottom surface of the harvester, e.g., on opposed sides of the orifice plate. The adhesive strips can be formed of a variety of different adhesive materials. Some examples of suitable adhesive materials can include, without limitation silicon. Generally, any suitable adhesive materials available in the art can be used.

In some embodiments, the device can include a strap for coupling it to a skin surface so as to securely place the distal end of the device adjacent to a skin portion. In some embodiments, the strap can be employed after the skin portion is stretched to ensure that the skin portion remains in a stretched state while a plurality of blisters are formed therein.

In some embodiments of the above device, the cutter can be in the form of a plate that includes a plurality of openings each of which is in alignment with one of the plurality of openings of the orifice plate when the cutter is assembled within the harvester.

In some embodiments, the openings in any of the orifice plate and the cutter plate can have a maximum dimension in a range of about 1 mm to about 12 mm, e.g., in a range of about 1 mm to about 10 mm, or in a range of about 2 mm to about 50 mm.

In some embodiments of the above method, the stretching of the skin portion can result in an increase in its surface area by a factor of at least about 1%, at least about 2%, at least about 5%, at least about 10%, at least about 12%, at least about 15%, or at least about 20%. Alternatively or additionally, the stretching of the skin portion can result in an increase in its surface area by a factor in a range varying from for example between at least about 1% to at least about 10%, between at least about 1% to at least about 20%, between at least about 2% to at least about 10%, or between at least about 2% to at least about 20%.

In some embodiments, a strap can be utilized to secure the device to a skin surface subsequent to the stretching of the skin portion and prior to the application of a negative pressure to the stretched skin to generate a plurality of blisters.

Other aspects and advantages of the invention can become apparent from the following drawings and description, all of which illustrate the. various aspects of the invention, by way of example only.

### BRIEF DESCRIPTION OF THE DRAWINGS

Features and advantages of the invention described herein, together with further advantages, may be better understood by referring to the following description taken in conjunction with the accompanying drawings. The drawings are not necessarily to scale, emphasis instead is generally placed upon illustrating the principles of the invention.

FIG. 1 is a flow chart depicting various steps in a method according to an embodiment for generating a plurality of skin blisters;

FIG. 2A is a view of the bottom portion of a device for generating skin grafts according to an embodiment, depicting an orifice plate as well as a skin-stretching mechanism including two slide rollers disposed on the opposed sides of the orifice plate;

FIG. 2B is a schematic side view of a harvester according to an embodiment of the present teachings;

FIG. 2C is a schematic top view of a device according to an embodiment for generating skin grafts, depicting a lid of the device;

FIG. 2D is a schematic side view of the skin-graft generating device depicted in FIG. 2C;

FIG. 3A is a schematic perspective view of an orifice plate, a cutter plate and an upper plate employed in an embodiment of a device for generating skin grafts according to the present teachings;

FIG. 3B is a partial view of the cutter plate depicted in FIG. 3A, which shows that each hole of the cutter plate includes a cutting edge for cutting a blister protruding therethrough;

FIG. 3C schematically depicts a plurality of frangible couplings that can be used to couple the cutter plate of a harvester according to the present teachings to any of the orifice and/or the upper plate;

FIG. 4 schematically depicts fixating a skin-graft generating device according to the present teachings to a patient's thigh via a strap;

FIG. 5A is a schematic perspective view of a harvester according to an embodiment of the present teachings in a blister-forming position;

FIG. 5B is a sectional side view of an actuating mechanism for moving the cutter plate in an initial position;

FIG. 5C is a perspective view of the harvester of FIG. 5A in a cocked position;

FIG. 5D is a sectional side view of the actuation mechanism for moving the cutter plate in cocked position;

FIG. 5E and FIG. 5F illustrate the handle of a harvester according to an embodiment in a mid-cut position;

FIG. 6A and FIG. 6B show a harvester according to an embodiment in a transitional position; and

FIG. 7 is a bottom view of a device for generating skin grafts according to an embodiment of the present teachings, which includes a plurality of adhesive strips for facilitating the stretching of a skin portion.

### DETAILED DESCRIPTION

The present disclosure relates generally to methods and devices for generating skin grafts, which employ stretching of a skin portion to facilitate the formation of a plurality of blisters in that portion.

In some embodiments, devices according to the present teachings for generating skin grafts incorporate a mechanism for stretching a skin portion and maintaining that skin portion in a stretched state as a plurality of blisters are formed in the stretched skin portion, e.g., by raising segments of the skin portion through one or more openings of an orifice plate coupled to a low-pressure chamber.

The term “adjacent to a portion of a subject's skin” as used herein is intended to mean in contact with or in close proximity to a portion of a subject's skin. The term a “skin-stretching mechanism” as used herein refers to a mechanism that allows stretching a skin portion and/or maintaining a stretched skin portion in a stretched state.

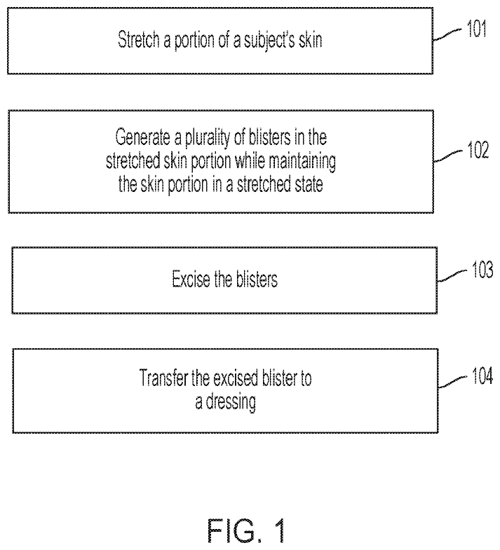

More particularly, with reference to the flow chart of FIG. 1, in one embodiment of a method according to the present teachings for generating skin grafts, a portion of a subject's skin is stretched (101) and subsequently, a plurality of blisters (e.g., epidermal blisters) is formed in the stretched skin portion while the skin portion is maintained in a stretched state (102). The blisters can then be harvested, e.g., by exciting the blisters (103) and transferring the blisters to a dressing (104), such as a silicone dressing. The harvested skin blisters can be transplanted onto a recipient site, e.g., a wound area. In some embodiments, the stretching of the skin portion can result in an increase in the surface area of that skin portion by a factor, for example, of at least about 1%, e.g., in a range of about 1% or 2% to about 10% or 20%.

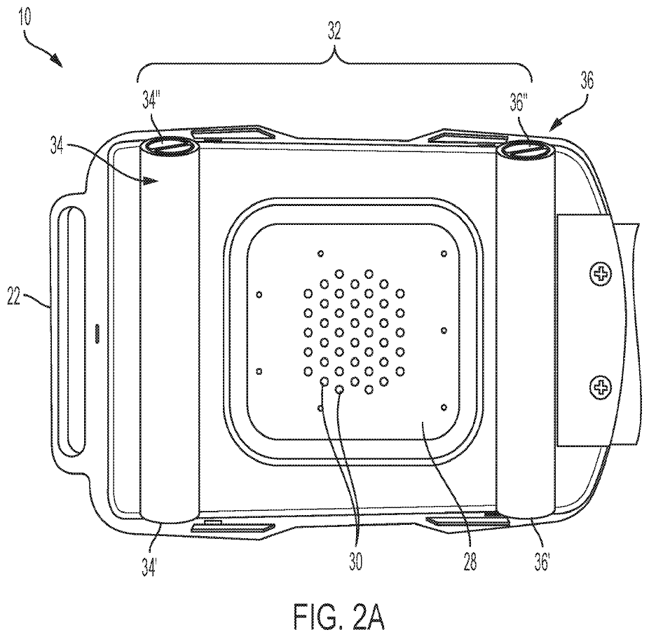

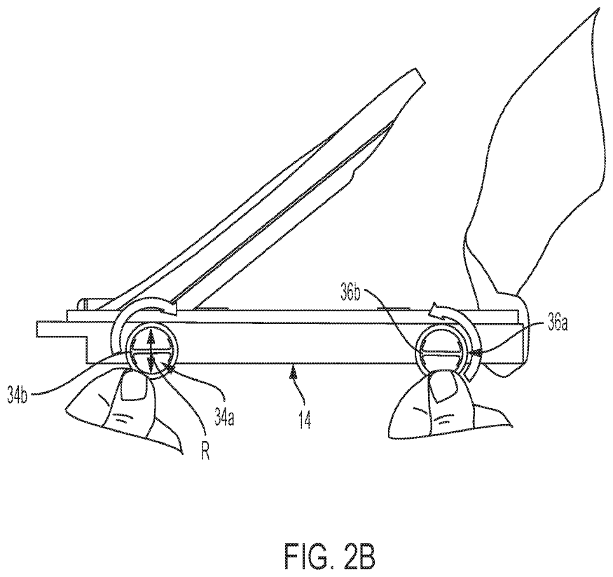

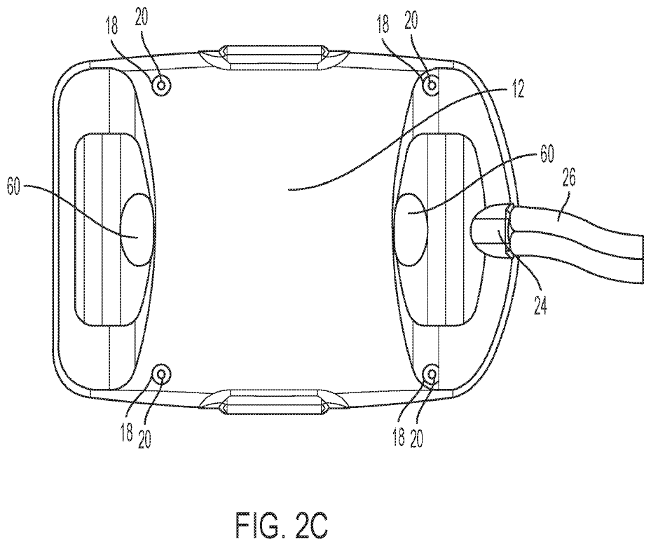

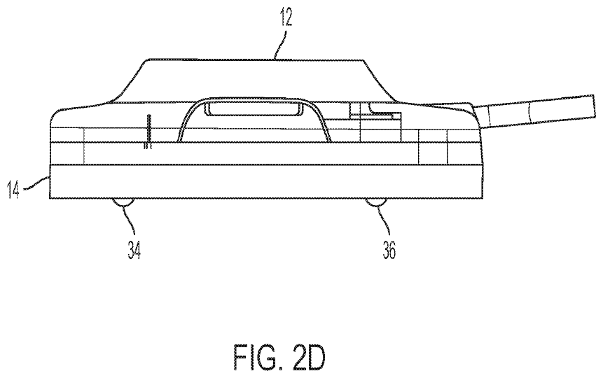

With reference to FIGS. 2A-2D, a device 10 according to an embodiment of the present teachings for generating skin grafts can include a head 12 (herein also referred to as a lid or a top element) that can be removably and replaceably attached to a harvester 14 to form a hollow chamber 16 into which a plurality of skin blisters can be drawn, as discussed below. The harvester 12 and the head 14 can be coupled to one another via one or more threaded screws 18 that are received by corresponding holes 20 in the lid and the body of the harvester 12. The harvester 12 can include a strap coupler 22 for joining a strap, e.g., a hook and fastener-type strap, to the device to facilitate attachment of the device to a patient, e.g., by wrapping the device around a patient's thigh for generating microblisters.

The head 12 includes a suction coupling 24 that allows coupling the head 12 via a suction tubing 26 to a vacuum source (not shown) to generate a negative pressure within the chamber.

Referring again to FIG. 2A, in this embodiment, the device 10 can further include an orifice plate 28 having a plurality of openings 30 through which a plurality of skin blisters can be raised into the hollow chamber of the device 10, as discussed in more detail below. Further, the openings 30 can have a circular shape with a diameter in a range of about 1 mm to about 10 mm, though other shapes and dimensions may also be employed.

The device 10 can also include a skin-stretching mechanism 32 for stretching a skin portion in which blisters are to be formed. In this embodiment, the skin-stretching mechanism can include two slide rollers 34 and 36, which are coupled to a distal end of the harvester 14 on opposed sides of the orifice plate 28. In this embodiment, each of the slide rollers 34/36 is in the form of a cylinder that extends from a proximal end 34′/36′ to a distal end 34″/36″. Each slide roller 34/36 is disposed in a respective cylindrical cavity such that a bottom portion of the slide roller is substantially flush with, or protrudes slightly beyond, the bottom surface of the harvester. Further, each slider roller 34/36 can include a knob 34a/36a at is proximal end that allows manual rotation of the roller. More specifically, the knobs 34a/36a can include finger-grip protrusions 34b/36b, respectively, that allow a user to rotate the slider rollers so as to stretch a skin portion disposed between the slide rollers.

In some embodiments, the slide rollers have a diameter R in a range of about 5 cm to about 20 cm, and a length that is substantially equal to the width of the bottom portion of the harvester body. Generally, any suitable diameter and length available in the art can be used for the slide rollers.

The outer surface of each slide roller is formed of a tacky biocompatible material. Some examples of suitable materials include, without limitation, silicon, acrylic, or a mix of silicon and acrylic in a pattern. Generally any suitable tacky biocompatible material available in the art can be used. In some embodiments, each slide roller includes a core formed of a plastic material on which a layer of the tacky biocompatible material is disposed. Alternatively, the slide rollers can be formed entirely of the tacky biocompatible material.

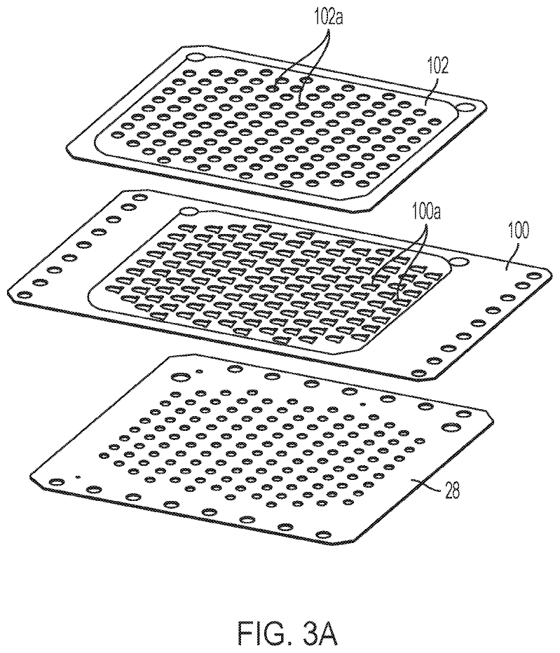

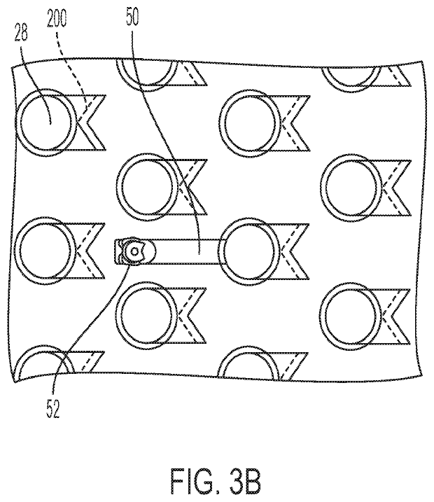

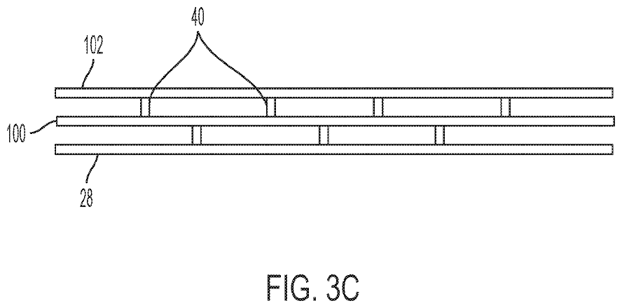

With reference to FIGS. 2A and 3A, the device 10 can further include a cutter plate 100 disposed in the harvester 14 for cutting blisters raised into the hollow chamber via the openings of the orifice plate 28. The cutter plate 100 is coupled to the orifice plate 28 and an upper plate 102 via a plurality of frangible linkages, as discussed in more detail below. The cutter plate 100 and the upper plate 102 can include a plurality of openings 100a/102a, which are in concentric alignment with one another as well as with the openings of the orifice plate when assembled in the harvester 14 such that a plurality of suction blisters can be raised through the openings of the orifice plate, the cutter plate and the upper plate.

In some embodiments, a plurality of coupling members, such as coupling members 40 depicted in FIG. 3C, are used to create frangible couplings between the cutter plate 100 and at least one of the orifice plate 28 and the upper plate 102. The coupling members can be disposed between the openings of the plates and/or in the outer perimeters thereof to form frangible couplings that can be broken upon lateral movement of the cutter plate with respect to the orifice plate and/or the upper plate. A variety of methods can be used to manufacture the plates and/or the coupling members, such as drilling, milling, laser etching, lithographic processing, photo etching, laser ablation and the like. For example, the coupling members can be frangibly coupled between the plates via spot welding, or via an adhesive such as epoxy. In some embodiments, a portion of the plate material at or around the site of a frangible coupling can be removed to accommodate at least a portion of the coupling member by forming a depression at or around the frangible section.

With reference to FIG. 3B, the cutter plate 100 can include one or more grooves or channels 50 that are configured to receive a pin 52 extending vertically from the orifice plate 28. The pin 52 is received at one end of channel 50 when the frangible couplings are intact, and laterally slides with the channel 50 to the opposite end when the frangible couplings are broken. Further details regarding the formation of the plates as well as frangible couplings can be found in U.S. Published Patent Application No. 2013/0204273, which is incorporated herein by reference in its entirety.

With reference to FIG. 3B, each of the cutter plate holes can provide a cutting edge 200 to partially occlude the respective opening and cleave a blister raised through the opening when the cutter plate is moved relative to the orifice plate 28 and the upper plate 102, as discussed in more detail below.

In use, the device 10 can be placed on a portion of a subject's skin with the slide rollers in contact with the skin and the orifice plate adjacent to a skin portion (e.g., in contact with the skin portion) between the two slide rollers. The slide rollers 34/36 can then be rotated in opposite directions (i.e., one clock-wise and the other counter clockwise), as shown schematically in FIG. 2B, to stretch the skin portion below the orifice plate. In some embodiments, the stretching of the skin portion beneath the orifice plate can result in an increase of at least about 1% or at least about 2% in its surface area, e.g., an increase in a range of about 1% or about 2% to about 10% or to about 20%.

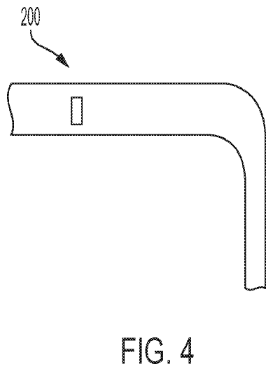

With reference to FIG. 1A as well as FIG. 4, with the skin portion beneath the orifice plate in a stretched state, a strap 200 attached to the strap coupler 22 can be used to fixate the device to the subject, e.g., to the subject's thigh with the orifice plate in proximity of the inner thigh. The strap can press the slide rollers against the skin and thereby facilitate maintaining the stretched skin portion in a stretched state.

The device can further be coupled to a vacuum source, via the vacuum coupling 24 in the lid 12 and the tube 26, to generate a negative pressure within the hollow chamber of the device, thereby raising a plurality of blisters through the openings in the orifice plate 28, the cutter plate 100 and the upper plate 102. In some embodiments, a heater incorporated in the device (e.g., in the lid) can be used to heat the orifice plate and hence the skin below the orifice plate to facilitate the formation of skin blisters. In some embodiments, the blisters can have a lateral dimension of less than about 2 mm, e.g., 100 to 2000 microns. With reference to FIG. 2C, in this embodiment, a viewing window 60 provided in the lid 12 allows the visual monitoring of the formation of the blisters.

It has been discovered that maintaining the skin portion below the orifice plate in a stretched state can expedite the formation of skin blisters therein, via the application of a negative pressure through the openings of the orifice plate to the skin portion. Consequently, the application of a stretching force to the skin can allow the formation of skin blisters suitable for transplantation in a time period significantly less than the time period required to form the blisters in absence of such a stretching force. For example, in some embodiments, the stretching of the skin can result in reducing the time period required to form blisters suitable for transplantation by at least about 20%, or at least about 30%, or at least about 40%, or at least about 50%, or at least about 60%, at least about 70%, or at least about 80%, or at least about 90%. In some embodiments, the stretching of the skin can reduce the required time for the formation of skin blisters by half, or one-third. This can in turn allow faster application of epidermal grafts to a patient.

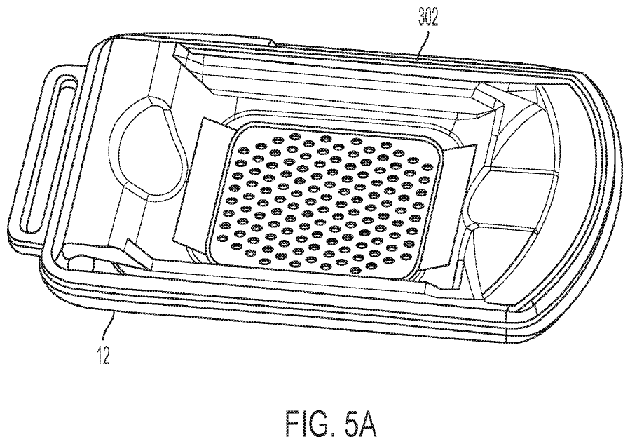

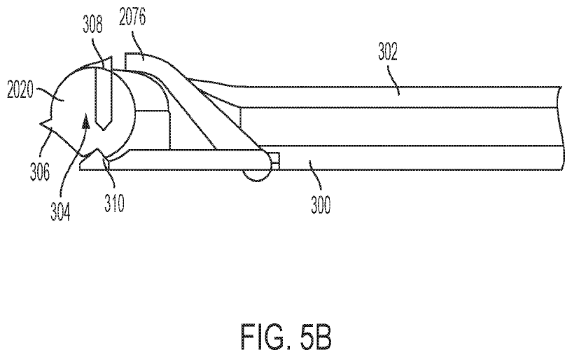

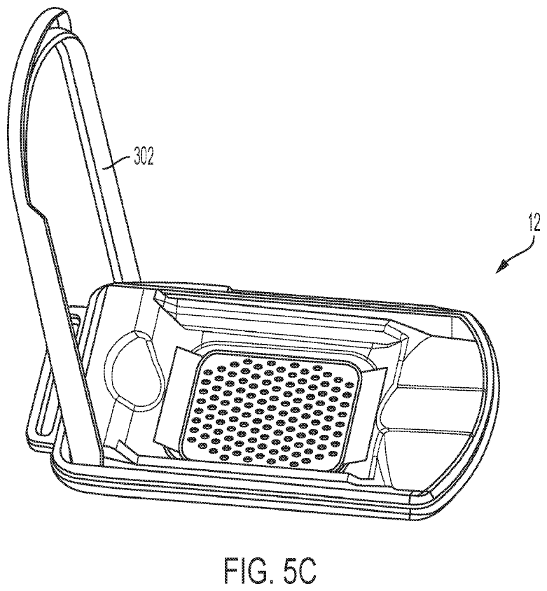

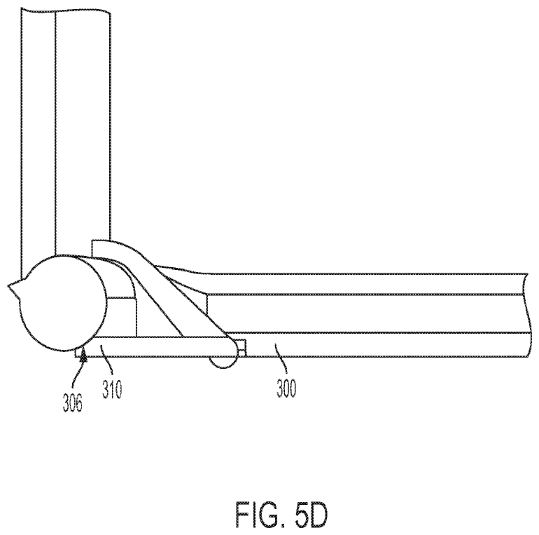

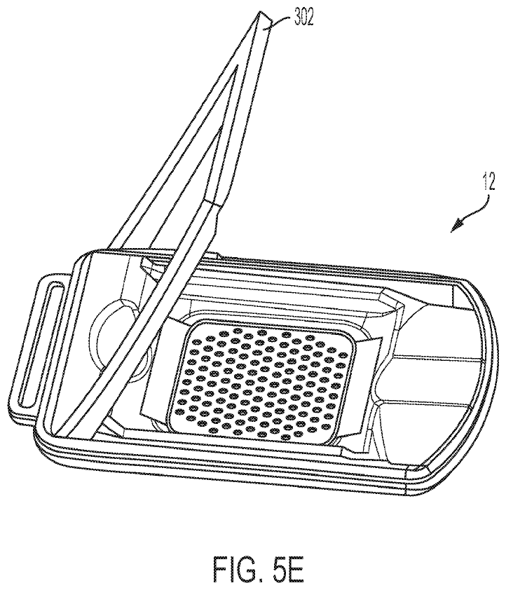

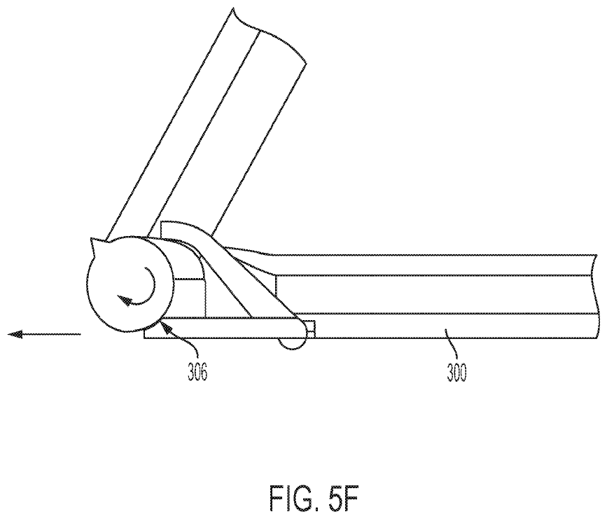

Once the blisters are formed, the application of a vacuum to the device can be stopped and the lid can be removed to provide access to the formed blisters. A dressing, such as a silicone dressing, can be placed over the blisters and the cutter plate can be moved via an actuator, discussed in more detail below, to cut the blisters. In particular, with reference to FIGS. 3A, 5A and 5B, the cutter plate 100 is joined to a sled 300 and a handle 302 is linked to the sled 300 via a generally cylindrical bar (axle) 304. As shown in FIG. 5B, the cylindrical bar 304 rotates about an axis when the handle is lifted up or closed. Cylindrical bar 304 also has two non-symmetric features: protrusions 306 and 308. The handle 302 can thu serves as a lever arm. As shown in FIGS. 5C and 5D, in a cocked position (handle up), the protrusion 306 (e.g., a longitudinal ridge on the cylindrical axle) engages with a mating feature 310 (e.g., a groove in the sled). As the handle is brought down the sled is thus forced to move by the rotation of the axle 304. FIGS. 5E and 5F illustrate the handle in a mid-cut position.

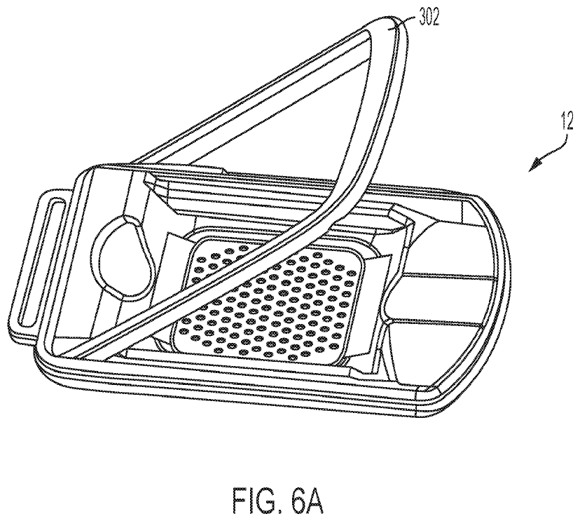

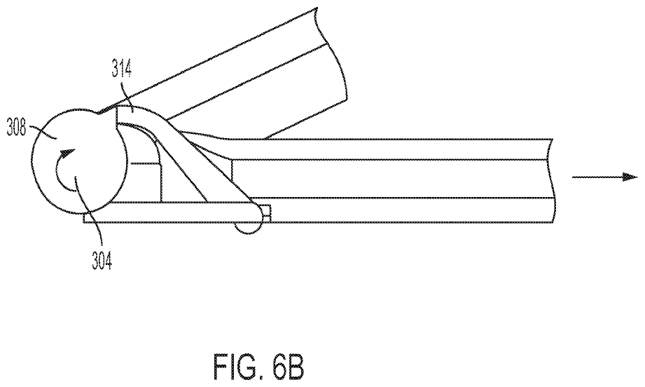

FIGS. 6A and 6B illustrate the harvester in a transitional position (from cutting to retraction). At this point in the rotation of the axle 304, protrusion 306 has detached from mating feature 310 and the second protrusion 308 has engaged with a different portion 314 (e.g., a shoulder pad) of the sled. As the handle continues its downward travel, the rotation of the axle causes the sled to move in the opposite direction and retract the cutter plate.

The cut blisters are captured by a dressing, e.g., a silicone dressing, and can be then transplanted onto a recipient site (e.g., a wound site).

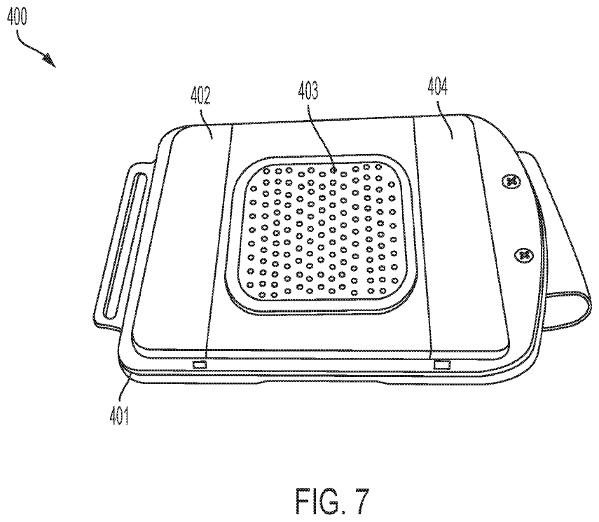

FIG. 7 schematically depicts another embodiment of a skin-graft generating device 400 according to the present teachings, which similar to the device 10 discussed above, includes a harvester 401 coupled to a lid (not shown in this figure). However, rather than utilizing a pair of slide rollers for stretching the skin, the skin-graft generating device 400 includes a pair of adhesive strips 402/404 that are disposed on the bottom portion of the harvester 401 on opposed sides of an orifice plate 403.

In use, a user can manually stretch a skin portion and then place the orifice plate in contact or in proximity of the stretched skin portion with the adhesive strips 402/404 positioned such that the stretched skin portion is maintained in a stretched state during the formation of blisters in a manner discussed above. In particular, the application of a vacuum to the device can raise a plurality of skin portions through the holes in the orifice plate, thereby generating a plurality of microblisters. The blisters can then be harvested in a manner discussed above.

Again, similar to the previous embodiment, maintaining the skin portion in which blisters are formed in a stretched state during the formation of the blisters can facilitate their formation and expedite the process of skin grafting.

Those having ordinary skill in the art will appreciate that various changes can be made to the above embodiments without departing from the scope of the invention. Although this specification discloses advantages in the context of certain illustrative, non-limiting embodiments, various changes, substitutions, permutations, and alterations may be made without departing from the scope of the specification as defined by the appended claims. Further, any feature described in connection with any one embodiment may also be applicable to any other embodiment.

What is claimed is:

1. A device for generating a skin graft, comprising:
a harvester having a distal end adapted to be disposed adjacent to a portion of a subject's skin, said harvester having an orifice plate coupled said distal end and having at least one opening;
a head configured for attachment to said harvester to form a chamber and configured for coupling to a vacuum source for generating a negative pressure in said chamber, thereby raising at least one blister through said at least one opening of the plate; and
a skin-stretching mechanism coupled to the harvester for stretching said skin portion;
wherein the harvester comprises a cutter integrated therein for cutting said blister.

2. The device of claim 1, wherein said skin-stretching mechanism is coupled to said distal end of the harvester.

3. The device of claim 1, wherein said stretching mechanism comprises a pair of slide rollers disposed on opposed sides of said orifice plate such that rotation of said rollers in opposite directions stretches said skin portion.

4. The device of claim 3, wherein each of said rollers comprises a tacky surface configured for placement on the subject's skin.

5. The device of claim 4, wherein said tacky surface comprises a biocompatible material.

6. The device of claim 3, wherein each of said rollers includes a finger-grip protrusion to allow manual rotation thereof.

7. The device of claim 3, wherein each of said rollers has a cylindrical shape extending between two ends.

8. The device of claim 7, wherein at least one end of each of said slide rollers comprises a finger-grip protrusion.

9. The device of claim 1, wherein said skin-stretching mechanism comprises a pair of adhesive strips disposed on opposed sides of said orifice plate.

10. The device of claim 1, further comprising a strap for coupling the device to a skin surface so as to place firmly said distal end of the harvester on or in proximity of said skin portion.

11. The device of claim 1, wherein said cutter comprises a plate having at least one opening in alignment with said at least one opening of the orifice plate when said cutter is assembled within said harvester.

12. The device of claim 1, wherein said at least one opening of the orifice plate has a maximum dimension in a range of about 1 mm to about 12 mm.

13. A method of generating a skin graft, comprising:
stretching a portion of a subject's skin;
placing a distal end of a device configured to generate one or more blisters adjacent to said skin portion, wherein said device comprises a negative pressure chamber and an orifice plate fitted to a distal end of said chamber and having one or more openings through which skin blisters can be raised into an interior of the chamber, and a cutter integrated for cutting the blisters;
applying a negative pressure to said chamber of the device to raise one or more blisters through one or more openings of said orifice plate while maintaining said skin portion in a stretched state; and
activating said cutter to cut the raised blisters.

14. The method of claim 13, wherein said step of stretching the skin portion results in an increase in a surface area of the skin portion by at least one of: a factor of at least about 1%, a factor of at least about 2%, a factor of at least about 5%, a factor of at least about 10%, a factor of at least about 15%, or a factor of at least about 20%.

15. The method of claim 13, further comprising utilizing a dressing to collect said cut blisters.

16. The method of claim 15, wherein said dressing comprises a silicone dressing.

17. The method of claim 13, further comprising utilizing a strap to secure said device to a skin surface prior to application of said negative pressure.

18. The method of claim 13, wherein the stretching of said skin portion facilitates formation of blisters therein.
